(19) United States
(12) Patent Application Publication
(10) Pub. No.: US 2022/0118133 A1
(43) Pub. Date: Apr. 21, 2022

(54) Combined UV Imaging and Sanitization

(71) Applicant: X Development LLC, Mountain View, CA (US)

(72) Inventors: Eden Rephaeli, Oakland, CA (US); Guy Satat, Sunnyvale, CA (US)

(21) Appl. No.: 17/450,173

(22) Filed: Oct. 7, 2021

Related U.S. Application Data
(60) Provisional application No. 63/093,692, filed on Oct. 19, 2020.

(51) Int. Cl. A61L 2/10; G06K 9/20; G06K 9/00

(52) U.S. Cl. CPC A61L 2/10; G06K 9/209; G06K 9/00664; A61L 2202/16; A61L 2202/11; A61L 2202/14; G06K 9/2027

(57) ABSTRACT

A system includes a robotic device, an ultraviolet (UV) illuminator disposed on the robotic device, an image sensor disposed on the robotic device and configured to sense UV light, and circuitry configured to perform operations. The operations include causing the UV illuminator to emit the UV light towards a feature of an environment, and receiving, from the image sensor, UV image data representing the feature illuminated by the UV light. The operations also include identifying, based on the UV image data, a portion of the feature to be sanitized by the robotic device, and based on the identifying the portion, adjusting a parameter of the UV illuminator from a first value associated with UV imaging to a second value associated with UV sanitization. The operations further include causing the robotic device to sanitize the portion of the feature by emitting, by the UV illuminator, the UV light towards the portion.

### CROSS-REFERENCE TO RELATED APPLICATIONS

This application claims priority to U.S. Provisional Patent Application No. 63/093,692, filed on Oct. 19, 2020, and titled “Combined UV Imaging and Sanitization,” which is hereby incorporated by reference as if fully set forth in this description.

### BACKGROUND

As technology advances, various types of robotic devices are being created for performing a variety of functions that may assist users. Robotic devices may be used for applications involving material handling, transportation, welding, assembly, and dispensing, among others. Over time, the manner in which these robotic systems operate is becoming more intelligent, efficient, and intuitive. As robotic systems become increasingly prevalent in numerous aspects of modern life, it is desirable for robotic systems to be efficient. Therefore, a demand for efficient robotic systems has helped open up a field of innovation in actuators, movement, sensing techniques, as well as component design and assembly.

### SUMMARY

A robotic device may use an ultraviolet (UV) illuminator to emit UV light towards different features of an environment in order to detect portions of these features to be sanitized, and to sanitize the detected portions. Specifically, the robotic device may include a UV sensor configured to generate UV image data representing reflections of the UV light from the features of the environment. The UV image data may represent various stains on the features of the environment that would not otherwise appear within image data captured using a different portion of the electromagnetic spectrum. Once a stain is identified, one or more parameters of the UV illuminator may be adjusted to configure the UV illuminator to deliver at least a target radiant exposure associated with effective sanitization of the portion. The UV illuminator may then be used to sanitize the feature by emitting additional UV light towards the feature. Once the portion is sanitized, the parameters of the UV illuminator may again be adjusted to values associated with UV imaging, rather than sanitization, and the operations may be repeated to identify and sanitize additional portions of the environment.

In a first example embodiment, a system may include a robotic device, a UV illuminator disposed on the robotic device and configured to emit UV light, an image sensor disposed on the robotic device and configured to sense the UV light, and circuitry configured to perform operations. The operations may include causing the UV illuminator to emit the UV light towards a feature of an environment, and receiving, from the image sensor, UV image data representing the feature illuminated by the UV light. The operations may also include identifying, based on the UV image data, a portion of the feature to be sanitized by the robotic device. The operations may additionally include, based on identifying the portion of the feature to be sanitized by the robotic device, adjusting at least one parameter of the UV illuminator from a first value associated with UV imaging to a second value associated with UV sanitization. The operations may further include, after adjusting the at least one parameter of the UV illuminator, causing the robotic device to sanitize the portion of the feature by emitting, by way of the UV illuminator, the UV light towards the portion of the feature.

In a second example embodiment, a method may include causing a UV illuminator disposed on a robotic device to emit UV light towards a feature of an environment. The method may also include receiving, from an image sensor disposed on the robotic device and configured to sense the UV light, UV image data representing the feature illuminated by the UV light. The method may additionally include identifying, based on the UV image data, a portion of the feature to be sanitized by the robotic device. The method may further include, based on identifying the portion of the feature to be sanitized by the robotic device, adjusting at least one parameter of the UV illuminator from a first value associated with UV imaging to a second value associated with UV sanitization. The method may yet further include, after adjusting the at least one parameter of the UV illuminator, causing the robotic device to sanitize the portion of the feature by emitting, by way of the UV illuminator, the UV light towards the portion of the feature.

In a third example embodiment, a non-transitory computer-readable medium may have stored thereon instructions that, when executed by a computing device, cause the computing device to perform operations. The operations may include causing a UV illuminator disposed on a robotic device to emit UV light towards a feature of an environment. The operations may also include receiving, from an image sensor disposed on the robotic device and configured to sense the UV light, UV image data representing the feature illuminated by the UV light. The operation may additionally include identifying, based on the UV image data, a portion of the feature to be sanitized by the robotic device. The operation may further include, based on identifying the portion of the feature to be sanitized by the robotic device, adjusting at least one parameter of the UV illuminator from a first value associated with UV imaging to a second value associated with UV sanitization. The operation may yet further include, after adjusting the at least one parameter of the UV illuminator, causing the robotic device to sanitize the portion of the feature by emitting, by way of the UV illuminator, the UV light towards the portion of the feature.

In a fourth example embodiment, a system may include means for causing a UV illuminator disposed on a robotic device to emit UV light towards a feature of an environment. The system may also include means for receiving, from an image sensor disposed on the robotic device and configured to sense the UV light, UV image data representing the feature illuminated by the UV light. The system may additionally include means for identifying, based on the UV image data, a portion of the feature to be sanitized by the robotic device. The system may further include means for, based on identifying the portion of the feature to be sanitized by the robotic device, adjusting at least one parameter of the UV illuminator from a first value associated with UV imaging to a second value associated with UV sanitization. The system may yet further include means for, after adjusting the at least one parameter of the UV illuminator, causing the robotic device to sanitize the portion of the feature by emitting, by way of the UV illuminator, the UV light towards the portion of the feature.

In a fifth example embodiment, a system may include a UV illuminator configured to emit UV light, an image sensor configured to sense the UV light, and circuitry configured to perform operations. The operations may include causing the UV illuminator to emit the UV light towards a feature of an environment, and receiving, from the image sensor, UV image data representing the feature illuminated by the UV light. The operations may also include identifying, based on the UV image data, a portion of the feature to be sanitized. The operations may additionally include, based on identifying the portion of the feature to be sanitized, adjusting at least one parameter of the UV illuminator from a first value associated with UV imaging to a second value associated with UV sanitization. The operation may further include, after adjusting the at least one parameter of the UV illuminator, causing the UV illuminator to emit the UV light towards the portion of the feature to sanitize the portion of the feature.

In a sixth example embodiment, a method may include causing a UV illuminator to emit UV light towards a feature of an environment. The method may also include receiving, from an image sensor configured to sense the UV light, UV image data representing the feature illuminated by the UV light. The method may additionally include identifying, based on the UV image data, a portion of the feature to be sanitized. The method may further include, based on identifying the portion of the feature to be sanitized, adjusting at least one parameter of the UV illuminator from a first value associated with UV imaging to a second value associated with UV sanitization. The method may yet further include, after adjusting the at least one parameter of the UV illuminator, causing the UV illuminator to emit the UV light towards the portion of the feature to sanitize the portion of the feature.

In a seventh example embodiment, a non-transitory computer-readable medium may have stored thereon instructions that, when executed by a computing device, cause the computing device to perform operations. The operations may include causing a UV illuminator to emit UV light towards a feature of an environment. The operations may also include receiving, from an image sensor configured to sense the UV light, UV image data representing the feature illuminated by the UV light. The operation may additionally include identifying, based on the UV image data, a portion of the feature to be sanitized. The operation may further include, based on identifying the portion of the feature to be sanitized, adjusting at least one parameter of the UV illuminator from a first value associated with UV imaging to a second value associated with UV sanitization. The operation may yet further include, after adjusting the at least one parameter of the UV illuminator, causing the UV illuminator to emit the UV light towards the portion of the feature to sanitize the portion of the feature.

In an eighth example embodiment, a system may include means for causing a UV illuminator to emit UV light towards a feature of an environment. The system may also include means for receiving, from an image sensor configured to sense the UV light, UV image data representing the feature illuminated by the UV light. The system may additionally include means for identifying, based on the UV image data, a portion of the feature to be sanitized. The system may further include means for, based on identifying the portion of the feature to be sanitized, adjusting at least one parameter of the UV illuminator from a first value associated with UV imaging to a second value associated with UV sanitization. The system may yet further include means for, after adjusting the at least one parameter of the UV illuminator, causing the UV illuminator to emit the UV light towards the portion of the feature to sanitize the portion of the feature.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 illustrates a configuration of a robotic system, in accordance with example embodiments.

FIG. 2 illustrates a mobile robot, in accordance with example embodiments.

FIG. 3 illustrates an exploded view of a mobile robot, in accordance with example embodiments.

FIG. 4 illustrates a robotic arm, in accordance with example embodiments.

FIG. 5A illustrates UV imaging of an environment, in accordance with example embodiments.

FIG. 5B illustrates contents of UV image data representing an environment, in accordance with example embodiments.

FIG. 5C illustrates UV sanitization of an environment, in accordance with example embodiments.

FIG. 6 illustrates a system, in accordance with example embodiments.

FIG. 7 illustrates graphs of parameter values of a UV illuminator, in accordance with example embodiments.

FIG. 8 illustrates a flow chart, in accordance with example embodiments.

FIG. 9 illustrates a flow chart, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example,” “exemplary,” and/or “illustrative” is not necessarily to be construed as preferred or advantageous over other embodiments or features unless stated as such. Thus, other embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order. Unless otherwise noted, figures are not drawn to scale.

### I. Overview

A robotic device may be configured to detect and/or clean features within an environment that appear dirty and/or are in fact dirty, such as surfaces have been touched by actors (e.g., humans or animals). Specifically, the robotic device may be configured to use an ultraviolet (UV) light source both to detect the features to be sanitized and to sanitize the detected features. Thus, the robotic device may include a UV illuminator configured to emit UV light, and a UV image sensor configured to sense the UV light as it reflects from features of the environment.

As the robotic device traverses the environment, the robotic device may use the UV illuminator to illuminate various features of the environment, and may use the UV image sensor to capture images of these various features illuminated by the UV light. The UV images may contain representations of various stains, residues, and/or other substances (collectively referred to herein as stains) which, in some cases, might not otherwise be apparent within images captured using other portions of the electromagnetic spectrum, such as using visible light. That is, the UV images may be used to find portions of features that are in fact dirty, but that might otherwise appear clean in images captured using non-UV light.

Control circuitry may be configured to receive the UV images, process the UV images to identify portions of features of the environment to be sanitized (e.g., disinfected, sterilized, and/or otherwise cleaned), and issue commands to the robotic device to perform the sanitization. The control circuitry may be physically located on the robotic device and/or remotely from the robotic device. Specifically, the control circuitry may be configured to identify, within the UV images, one or more visual patterns (e.g., blobs and/or arrangements thereof) that may represent a stain, dirt, a residue, a foreign substance, or another undesirable material (collectively referred to herein as a stain) present on a feature of the environment. For example, the visual patterns may correspond to fingerprints, hand prints, paw prints, and/or prints left behind by dishware or cutlery, among other possibilities. In some cases, the one or more patterns may be identified, for example, by way of one or more machine learning models.

Based on identifying one or more such visual patterns within the UV images, the control circuitry may be configured to designate for sanitization at least a portion of the feature on which these patterns appear. For example, the control circuitry may update a subset of a map corresponding to the portion of the feature to indicate that the portion of the feature is determined to be dirty, contaminated, and/or otherwise not at a desired level of cleanliness. The control circuitry may be further configured to adjust at least one parameter associated with the UV illuminator from a first value associated with UV imaging to a second value associated with UV sanitization. That is, the control circuitry may reconfigure the UV illuminator to adapt it to be used to sanitize the feature, rather than illuminate the feature for imaging.

In one example, the parameter associated with the UV illuminator may be a position of the UV illuminator relative to the portion of the feature (e.g., measured along a dimension perpendicular to the portion of the feature). As the environment is being imaged, the UV illuminator may be positioned further away from the feature to capture a wider field of view that includes regions of the environment surrounding the feature. Once the portion of the feature is identified for sanitization, the UV illuminator may be brought closer to the portion of the feature so as to achieve a target irradiance (i.e., power per unit area) associated with successful and/or optimal sanitization of microorganisms.

In another example, the parameter associated with the UV illuminator may be a movement speed of the UV illuminator relative to the portion of the feature (e.g., measured along a dimension parallel to the portion of the feature). As the environment is being imaged, the UV illuminator may be moved relative to the feature at a first speed that allows the UV image sensor to capture UV image data representing different portions of the environment. Once the portion of the feature is identified for sanitization, the UV illuminator may be moved relative to the portion of the feature at a second (e.g., lower) speed so as to deliver a target radian exposure (i.e., energy per unit area) associated with successful and/or optimal sanitization of microorganisms.

In a further example, the parameter associated with the UV illuminator may be a power with which the UV illuminator emits the UV light. As the environment is being imaged, the UV illuminator may be configured to emit UV light with a power level that is sufficient for imaging of the environment, but that might not provide sufficient irradiance and/or radiant exposure to perform the sanitization. Thus, once the portion of the feature is identified for sanitization, the UV illuminator may be configured to emit UV light with a relatively higher power level that is sufficient to achieve the target irradiance and/or radiant exposure. Since the irradiance and radiant exposure depend on both the power level of the UV illuminator and the position thereof relative to the feature, these two parameters may be jointly adjusted to achieve the target irradiance and/or radiant exposure.

In a yet further example, the parameter associated with the UV illuminator may be a frequency range of the UV light. Specifically, a first subset of the UV wavelength spectrum may be used for imaging because UV light in the first subset of the spectrum may result in UV images that more clearly (e.g., with a greater contrast) represent stains within the environment. On the other hand, a second subset of the UV wavelength spectrum may be used for sanitization because UV light in the second subset of the spectrum may be relatively more germicidal. Specifically, germicidal properties of the UV light may peak between 255 and 275 nanometers, whereas UV light outside of this range may be better adapted to generating UV images representative of stains. Thus, for example, UV light between 200 and 300 nanometers may be used for sanitization, and UV light between 300 and 400 nanometers may be used for imaging.

In some implementations, when capturing UV images, the wavelength of the light may be varied from 200 nanometers to 400 in, for example, 25 nanometer increments, thus allowing for multiple UV images to be captured, each representative of a different wavelength. By processing multiple UV images at different wavelengths, the control circuitry may be able to detect stains that appear at a first UV frequency, but that might not appear at other UV frequencies.

Once the UV illuminator is adapted for sanitization, the control circuitry may cause the robotic device to sanitize the portion of the feature by using the UV illuminator to emit the UV light towards the feature. Depending on the size of the portion of the feature and/or the position of the feature relative to the robotic device, the robotic device may be repositioned (e.g., at the second speed) to scan the portion of the feature using the UV light, thereby sanitizing the entirety thereof In some implementations, the UV illuminator may be connected to an arm of the robotic device, and may thus be repositioned relative to the environment by controlling the arm. Further, in some implementations, the arm may also include thereon an end effector configured to physically clean the portion of the feature prior to sanitization thereof by the UV light. Thus, the end effector may remove from the feature any physical debris under which microorganisms may be present, thereby exposing these microorganisms to the UV light.

Once the sanitization of the feature is completed, the parameters of the UV light may be adjusted back to the values associated with imaging, rather than sanitization. In some cases, the map of the environment may be updated to indicate a time at which the portion of the environment was sanitized, so that when the same stains are again detected on the portion at a future time (e.g., within a threshold amount of time of the initial sanitization), these same stains might not be resanitized. As additional stains are detected within additional UV images, the process may be repeated. Thus, a single UV illuminator may be used both for imaging of the environment and sanitization of the environment by adjusting the parameters thereof in accordance with the operating being performed.

In some implementations, the robotic device may also include additional sensors configured to detect light in other parts of the electromagnetic spectrum, such as visible light or near-infrared (NIR) light, and/or configured to sense depth. The control circuitry may be configured to similarly process images representative of these other spectra and/or depth to detect therein one or more stains, and/or assist with detecting stains in the UV image data. The control circuitry may also be configured to combine the various stains detected across the different spectra, and mark each of these stains for sanitization. Thus, in addition to emitting UV light toward stains visible in the UV spectrum, the robotic device may also emit UV light toward stains visible in other spectra, thereby allowing for a more thorough sanitization of features of the environment. The UV image sensor and/or the additional sensors may be positioned on the arm of the robotic device, and may thus be collocated with the UV illuminator, and/or on a body of the robotic device. In some cases, the systems and operations described herein may be performed without involving a robotic device.

### II. Example Robotic Systems

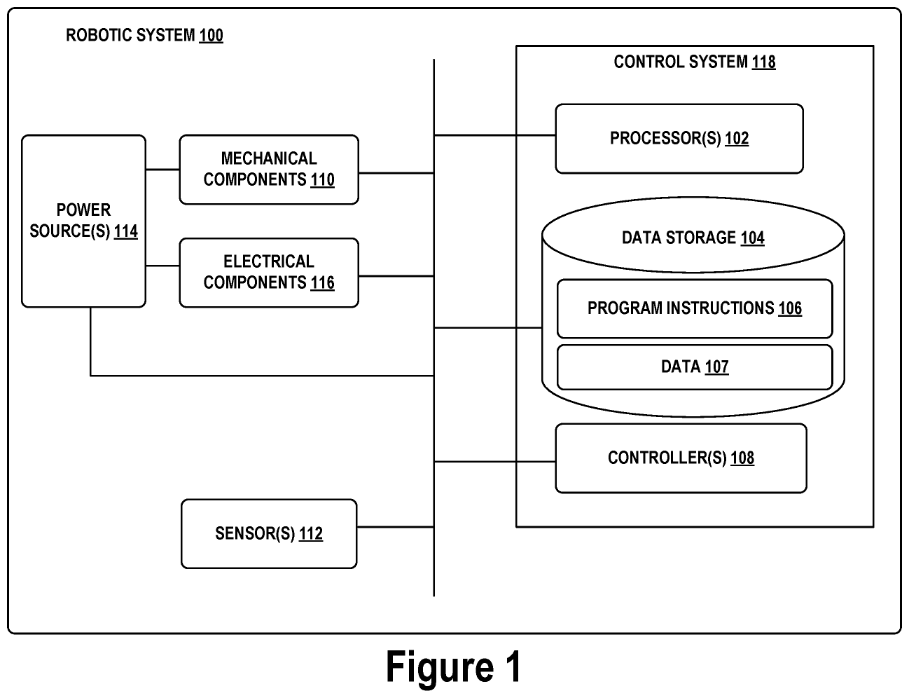

FIG. 1 illustrates an example configuration of a robotic system that may be used in connection with the implementations described herein. Robotic system 100 may be configured to operate autonomously, semi-autonomously, or using directions provided by user(s). Robotic system 100 may be implemented in various forms, such as a robotic arm, industrial robot, or some other arrangement. Some example implementations involve a robotic system 100 engineered to be low cost at scale and designed to support a variety of tasks. Robotic system 100 may be designed to be capable of operating around people. Robotic system 100 may also be optimized for machine learning. Throughout this description, robotic system 100 may also be referred to as a robot, robotic device, and/or mobile robot, among other designations.

As shown in FIG. 1, robotic system 100 may include processor(s) 102, data storage 104, and controller(s) 108, which together may be part of control system 118. Robotic system 100 may also include sensor(s) 112, power source(s) 114, mechanical components 110, and electrical components 116. Nonetheless, robotic system 100 is shown for illustrative purposes, and may include more or fewer components. The various components of robotic system 100 may be connected in any manner, including by way of wired or wireless connections. Further, in some examples, components of robotic system 100 may be distributed among multiple physical entities rather than a single physical entity. Other example illustrations of robotic system 100 may exist as well.

Processor(s) 102 may operate as one or more general-purpose hardware processors or special purpose hardware processors (e.g., digital signal processors, application specific integrated circuits, etc.). Processor(s) 102 may be configured to execute computer-readable program instructions 106 and manipulate data 107, both of which are stored in data storage 104. Processor(s) 102 may also directly or indirectly interact with other components of robotic system 100, such as sensor(s) 112, power source(s) 114, mechanical components 110, or electrical components 116.

Data storage 104 may be one or more types of hardware memory. For example, data storage 104 may include or take the form of one or more computer-readable storage media that can be read or accessed by processor(s) 102. The one or more computer-readable storage media can include volatile and/or non-volatile storage components, such as optical, magnetic, organic, or another type of memory or storage, which can be integrated in whole or in part with processor(s) 102. In some implementations, data storage 104 can be a single physical device. In other implementations, data storage 104 can be implemented using two or more physical devices, which may communicate with one another via wired or wireless communication. As noted previously, data storage 104 may include the computer-readable program instructions 106 and data 107. Data 107 may be any type of data, such as configuration data, sensor data, or diagnostic data, among other possibilities.

Controller 108 may include one or more electrical circuits, units of digital logic, computer chips, or microprocessors that are configured to (perhaps among other tasks), interface between any combination of mechanical components 110, sensor(s) 112, power source(s) 114, electrical components 116, control system 118, or a user of robotic system 100. In some implementations, controller 108 may be a purpose-built embedded device for performing specific operations with one or more subsystems of robotic system 100.

Control system 118 may monitor and physically change the operating conditions of robotic system 100. In doing so, control system 118 may serve as a link between portions of robotic system 100, such as between mechanical components 110 or electrical components 116. In some instances, control system 118 may serve as an interface between robotic system 100 and another computing device. Further, control system 118 may serve as an interface between robotic system 100 and a user. In some instances, control system 118 may include various components for communicating with robotic system 100, including a joystick, buttons, or ports, etc. The example interfaces and communications noted above may be implemented via a wired or wireless connection, or both. Control system 118 may perform other operations for robotic system 100 as well.

During operation, control system 118 may communicate with other systems of robotic system 100 via wired and/or wireless connections, and may further be configured to communicate with one or more users of the robot. As one possible illustration, control system 118 may receive an input (e.g., from a user or from another robot) indicating an instruction to perform a requested task, such as to pick up and move an object from one location to another location. Based on this input, control system 118 may perform operations to cause the robotic system 100 to make a sequence of movements to perform the requested task. As another illustration, a control system may receive an input indicating an instruction to move to a requested location. In response, control system 118 (perhaps with the assistance of other components or systems) may determine a direction and speed to move robotic system 100 through an environment en route to the requested location.

Operations of control system 118 may be carried out by processor(s) 102. Alternatively, these operations may be carried out by controller(s) 108, or a combination of processor(s) 102 and controller(s) 108. In some implementations, control system 118 may partially or wholly reside on a device other than robotic system 100, and therefore may at least in part control robotic system 100 remotely.

Mechanical components 110 represent hardware of robotic system 100 that may enable robotic system 100 to perform physical operations. As a few examples, robotic system 100 may include one or more physical members, such as an arm, an end effector, a head, a neck, a torso, a base, and wheels. The physical members or other parts of robotic system 100 may further include actuators arranged to move the physical members in relation to one another. Robotic system 100 may also include one or more structured bodies for housing control system 118 or other components, and may further include other types of mechanical components. The particular mechanical components 110 used in a given robot may vary based on the design of the robot, and may also be based on the operations or tasks the robot may be configured to perform.

In some examples, mechanical components 110 may include one or more removable components. Robotic system 100 may be configured to add or remove such removable components, which may involve assistance from a user or another robot. For example, robotic system 100 may be configured with removable end effectors or digits that can be replaced or changed as needed or desired. In some implementations, robotic system 100 may include one or more removable or replaceable battery units, control systems, power systems, bumpers, or sensors. Other types of removable components may be included within some implementations.

Robotic system 100 may include sensor(s) 112 arranged to sense aspects of robotic system 100. Sensor(s) 112 may include one or more force sensors, torque sensors, velocity sensors, acceleration sensors, position sensors, proximity sensors, motion sensors, location sensors, load sensors, temperature sensors, touch sensors, depth sensors, ultrasonic range sensors, infrared sensors, object sensors, or cameras, among other possibilities. Within some examples, robotic system 100 may be configured to receive sensor data from sensors that are physically separated from the robot (e.g., sensors that are positioned on other robots or located within the environment in which the robot is operating).

Sensor(s) 112 may provide sensor data to processor(s) 102 (perhaps by way of data 107) to allow for interaction of robotic system 100 with its environment, as well as monitoring of the operation of robotic system 100. The sensor data may be used in evaluation of various factors for activation, movement, and deactivation of mechanical components 110 and electrical components 116 by control system 118. For example, sensor(s) 112 may capture data corresponding to the terrain of the environment or location of nearby objects, which may assist with environment recognition and navigation.

In some examples, sensor(s) 112 may include RADAR (e.g., for long-range object detection, distance determination, or speed determination), LIDAR (e.g., for short-range object detection, distance determination, or speed determination), SONAR (e.g., for underwater object detection, distance determination, or speed determination), VICON® (e.g., for motion capture), one or more cameras (e.g., stereoscopic cameras for 3D vision), a global positioning system (GPS) transceiver, or other sensors for capturing information of the environment in which robotic system 100 is operating. Sensor(s) 112 may monitor the environment in real time, and detect obstacles, elements of the terrain, weather conditions, temperature, or other aspects of the environment. In another example, sensor(s) 112 may capture data corresponding to one or more characteristics of a target or identified object, such as a size, shape, profile, structure, or orientation of the object.

Further, robotic system 100 may include sensor(s) 112 configured to receive information indicative of the state of robotic system 100, including sensor(s) 112 that may monitor the state of the various components of robotic system 100. Sensor(s) 112 may measure activity of systems of robotic system 100 and receive information based on the operation of the various features of robotic system 100, such as the operation of an extendable arm, an end effector, or other mechanical or electrical features of robotic system 100. The data provided by sensor(s) 112 may enable control system 118 to determine errors in operation as well as monitor overall operation of components of robotic system 100.

As an example, robotic system 100 may use force/torque sensors to measure load on various components of robotic system 100. In some implementations, robotic system 100 may include one or more force/torque sensors on an arm or end effector to measure the load on the actuators that move one or more members of the arm or end effector. In some examples, the robotic system 100 may include a force/torque sensor at or near the wrist or end effector, but not at or near other joints of a robotic arm. In further examples, robotic system 100 may use one or more position sensors to sense the position of the actuators of the robotic system. For instance, such position sensors may sense states of extension, retraction, positioning, or rotation of the actuators on an arm or end effector.

As another example, sensor(s) 112 may include one or more velocity or acceleration sensors. For instance, sensor(s) 112 may include an inertial measurement unit (IMU). The IMU may sense velocity and acceleration in the world frame, with respect to the gravity vector. The velocity and acceleration sensed by the IMU may then be translated to that of robotic system 100 based on the location of the IMU in robotic system 100 and the kinematics of robotic system 100.

Robotic system 100 may include other types of sensors not explicitly discussed herein. Additionally or alternatively, the robotic system may use particular sensors for purposes not enumerated herein.

Robotic system 100 may also include one or more power source(s) 114 configured to supply power to various components of robotic system 100. Among other possible power systems, robotic system 100 may include a hydraulic system, electrical system, batteries, or other types of power systems. As an example illustration, robotic system 100 may include one or more batteries configured to provide charge to components of robotic system 100. Some of mechanical components 110 or electrical components 116 may each connect to a different power source, may be powered by the same power source, or be powered by multiple power sources.

Any type of power source may be used to power robotic system 100, such as electrical power or a gasoline engine. Additionally or alternatively, robotic system 100 may include a hydraulic system configured to provide power to mechanical components 110 using fluid power. Components of robotic system 100 may operate based on hydraulic fluid being transmitted throughout the hydraulic system to various hydraulic motors and hydraulic cylinders, for example. The hydraulic system may transfer hydraulic power by way of pressurized hydraulic fluid through tubes, flexible hoses, or other links between components of robotic system 100. Power source(s) 114 may charge using various types of charging, such as wired connections to an outside power source, wireless charging, combustion, or other examples.

Electrical components 116 may include various mechanisms capable of processing, transferring, or providing electrical charge or electric signals. Among possible examples, electrical components 116 may include electrical wires, circuitry, or wireless communication transmitters and receivers to enable operations of robotic system 100. Electrical components 116 may interwork with mechanical components 110 to enable robotic system 100 to perform various operations. Electrical components 116 may be configured to provide power from power source(s) 114 to the various mechanical components 110, for example. Further, robotic system 100 may include electric motors. Other examples of electrical components 116 may exist as well.

Robotic system 100 may include a body, which may connect to or house appendages and components of the robotic system. As such, the structure of the body may vary within examples and may further depend on particular operations that a given robot may have been designed to perform. For example, a robot developed to carry heavy loads may have a wide body that enables placement of the load. Similarly, a robot designed to operate in tight spaces may have a relatively tall, narrow body. Further, the body or the other components may be developed using various types of materials, such as metals or plastics. Within other examples, a robot may have a body with a different structure or made of various types of materials.

The body or the other components may include or carry sensor(s) 112. These sensors may be positioned in various locations on the robotic system 100, such as on a body, a head, a neck, a base, a torso, an arm, or an end effector, among other examples.

Robotic system 100 may be configured to carry a load, such as a type of cargo that is to be transported. In some examples, the load may be placed by the robotic system 100 into a bin or other container attached to the robotic system 100. The load may also represent external batteries or other types of power sources (e.g., solar panels) that the robotic system 100 may utilize. Carrying the load represents one example use for which the robotic system 100 may be configured, but the robotic system 100 may be configured to perform other operations as well.

As noted above, robotic system 100 may include various types of appendages, wheels, end effectors, gripping devices and so on. In some examples, robotic system 100 may include a mobile base with wheels, treads, or some other form of locomotion. Additionally, robotic system 100 may include a robotic arm or some other form of robotic manipulator. In the case of a mobile base, the base may be considered as one of mechanical components 110 and may include wheels, powered by one or more of actuators, which allow for mobility of a robotic arm in addition to the rest of the body.

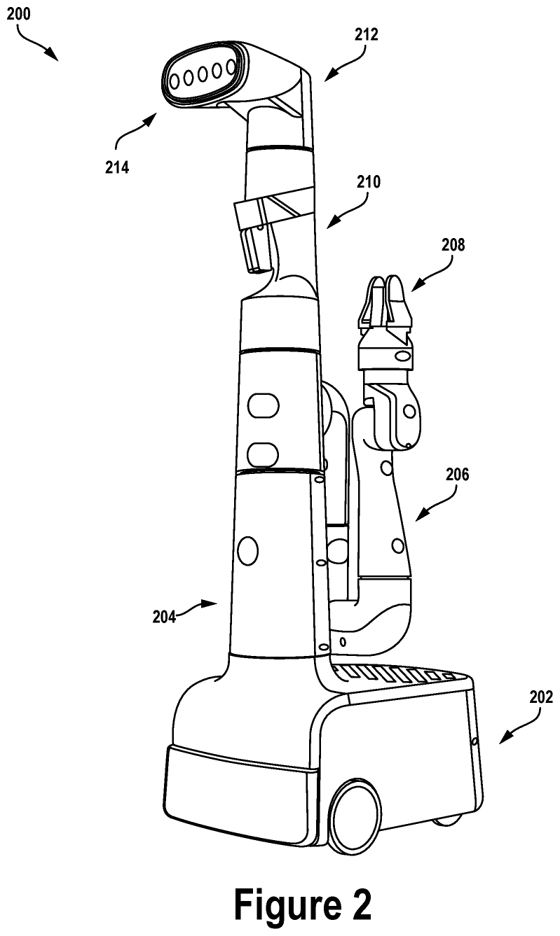

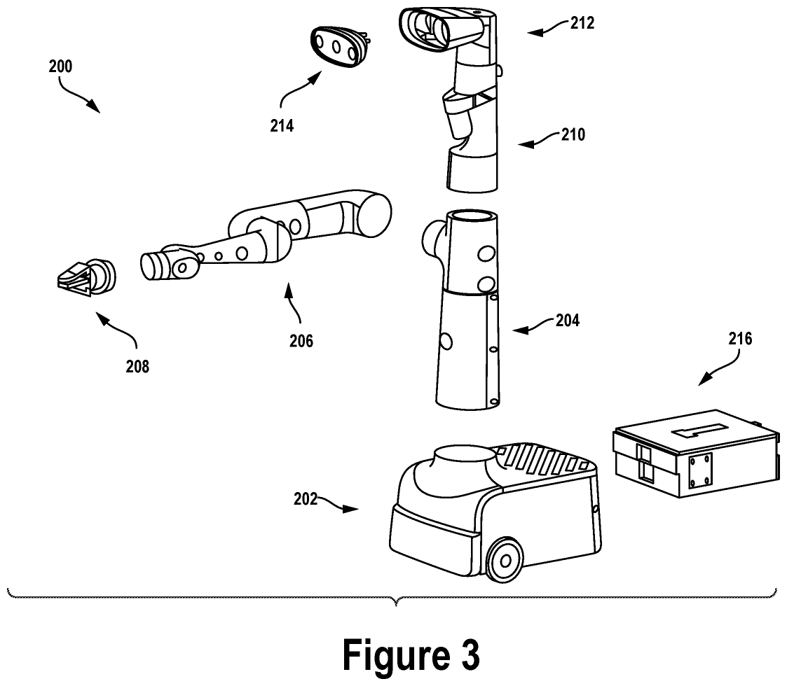

FIG. 2 illustrates a mobile robot, in accordance with example embodiments. FIG. 3 illustrates an exploded view of the mobile robot, in accordance with example embodiments. More specifically, robot 200 may include mobile base 202, midsection 204, arm 206, end-of-arm system (EOAS) 208, mast 210, perception housing 212, and perception suite 214. Robot 200 may also include compute box 216 stored within mobile base 202.

Mobile base 202 includes two drive wheels positioned at a front end of robot 200 in order to provide locomotion to robot 200. Mobile base 202 also includes additional casters (not shown) to facilitate motion of mobile base 202 over a ground surface. Mobile base 202 may have a modular architecture that allows compute box 216 to be easily removed. Compute box 216 may serve as a removable control system for robot 200 (rather than a mechanically integrated control system). After removing external shells, compute box 216 can be easily removed and/or replaced. Mobile base 202 may also be designed to allow for additional modularity. For example, mobile base 202 may also be designed so that a power system, a battery, and/or external bumpers can all be easily removed and/or replaced.

Midsection 204 may be attached to mobile base 202 at a front end of mobile base 202. Midsection 204 includes a mounting column which is fixed to mobile base 202. Midsection 204 additionally includes a rotational joint for arm 206. More specifically, Midsection 204 includes the first two degrees of freedom for arm 206 (a shoulder yaw J0 joint and a shoulder pitch J1 joint). The mounting column and the shoulder yaw J0 joint may form a portion of a stacked tower at the front of mobile base 202. The mounting column and the shoulder yaw J0 joint may be coaxial. The length of the mounting column of midsection 204 may be chosen to provide arm 206 with sufficient height to perform manipulation tasks at commonly encountered height levels (e.g., coffee table top and/or counter top levels). The length of the mounting column of midsection 204 may also allow the shoulder pitch J1 joint to rotate arm 206 over mobile base 202 without contacting mobile base 202.

Arm 206 may be a 7 DOF robotic arm when connected to midsection 204. As noted, the first two DOFs of arm 206 may be included in midsection 204. The remaining five DOFs may be included in a standalone section of arm 206 as illustrated in FIGS. 2 and 3. Arm 206 may be made up of plastic monolithic link structures. Inside arm 206 may be housed standalone actuator modules, local motor drivers, and thru bore cabling.

EOAS 208 may be an end effector at the end of arm 206. EOAS 208 may allow robot 200 to manipulate objects in the environment. As shown in FIGS. 2 and 3, EOAS 208 may be a gripper, such as an underactuated pinch gripper. The gripper may include one or more contact sensors such as force/torque sensors and/or non-contact sensors such as one or more cameras to facilitate object detection and gripper control. EOAS 208 may also be a different type of gripper such as a suction gripper or a different type of tool such as a drill or a brush. EOAS 208 may also be swappable or include swappable components such as gripper digits.

Mast 210 may be a relatively long, narrow component between the shoulder yaw JO joint for arm 206 and perception housing 212. Mast 210 may be part of the stacked tower at the front of mobile base 202. Mast 210 may be fixed relative to mobile base 202. Mast 210 may be coaxial with midsection 204. The length of mast 210 may facilitate perception by perception suite 214 of objects being manipulated by EOAS 208. Mast 210 may have a length such that when the shoulder pitch J1 joint is rotated vertical up, a topmost point of a bicep of arm 206 is approximately aligned with a top of mast 210. The length of mast 210 may then be sufficient to prevent a collision between perception housing 212 and arm 206 when the shoulder pitch J1 joint is rotated vertical up.

As shown in FIGS. 2 and 3, mast 210 may include a 3D lidar sensor configured to collect depth information about the environment. The 3D lidar sensor may be coupled to a carved-out portion of mast 210 and fixed at a downward angle. The lidar position may be optimized for localization, navigation, and for front cliff detection.

Perception housing 212 may include at least one sensor making up perception suite 214. Perception housing 212 may be connected to a pan/tilt control to allow for reorienting of perception housing 212 (e.g., to view objects being manipulated by EOAS 208). Perception housing 212 may be a part of the stacked tower fixed to mobile base 202. A rear portion of perception housing 212 may be coaxial with mast 210.

Perception suite 214 may include a suite of sensors configured to collect sensor data representative of the environment of robot 200. Perception suite 214 may include an infrared(IR)-assisted stereo depth sensor. Perception suite 214 may additionally include a wide-angled red-green-blue (RGB) camera for human-robot interaction and context information. Perception suite 214 may additionally include a high resolution RGB camera for object classification. A face light ring surrounding perception suite 214 may also be included for improved human-robot interaction and scene illumination. In some examples, perception suite 214 may also include a projector configured to project images and/or video into the environment.

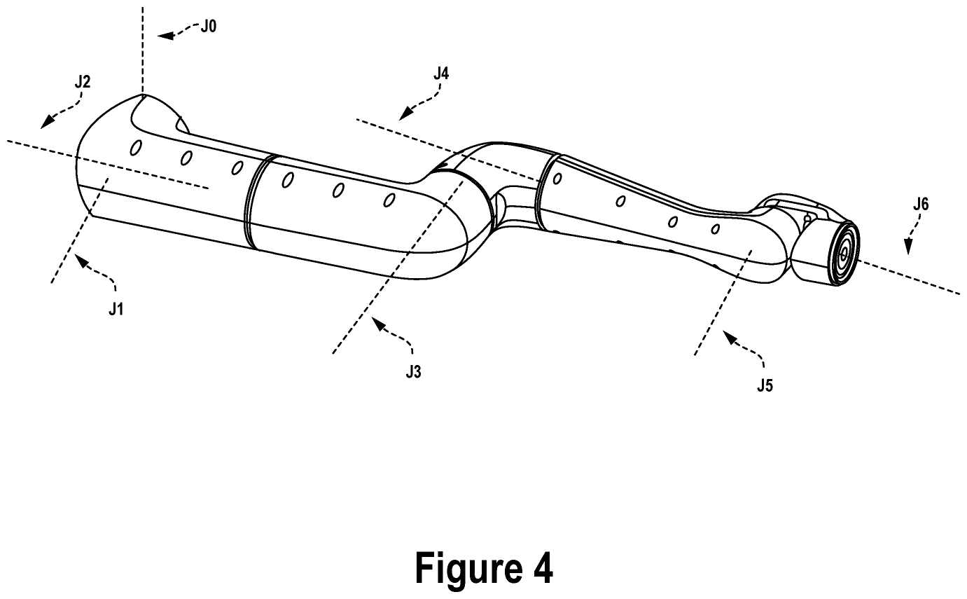

FIG. 4 illustrates a robotic arm, in accordance with example embodiments. The robotic arm includes 7 DOFs: a shoulder yaw J0 joint, a shoulder pitch J1 joint, a bicep roll J2 joint, an elbow pitch J3 joint, a forearm roll J4 joint, a wrist pitch J5 joint, and wrist roll J6 joint. Each of the joints may be coupled to one or more actuators. The actuators coupled to the joints may be operable to cause movement of links down the kinematic chain (as well as any end effector attached to the robot arm).

The shoulder yaw J0 joint allows the robot arm to rotate toward the front and toward the back of the robot. One beneficial use of this motion is to allow the robot to pick up an object in front of the robot and quickly place the object on the rear section of the robot (as well as the reverse motion). Another beneficial use of this motion is to quickly move the robot arm from a stowed configuration behind the robot to an active position in front of the robot (as well as the reverse motion).

The shoulder pitch J1 joint allows the robot to lift the robot arm (e.g., so that the bicep is up to perception suite level on the robot) and to lower the robot arm (e.g., so that the bicep is just above the mobile base). This motion is beneficial to allow the robot to efficiently perform manipulation operations (e.g., top grasps and side grasps) at different target height levels in the environment. For instance, the shoulder pitch J1 joint may be rotated to a vertical up position to allow the robot to easily manipulate objects on a table in the environment. The shoulder pitch J1 joint may be rotated to a vertical down position to allow the robot to easily manipulate objects on a ground surface in the environment.

The bicep roll J2 joint allows the robot to rotate the bicep to move the elbow and forearm relative to the bicep. This motion may be particularly beneficial for facilitating a clear view of the EOAS by the robot's perception suite. By rotating the bicep roll J2 joint, the robot may kick out the elbow and forearm to improve line of sight to an object held in a gripper of the robot.

Moving down the kinematic chain, alternating pitch and roll joints (a shoulder pitch J1 joint, a bicep roll J2 joint, an elbow pitch J3 joint, a forearm roll J4 joint, a wrist pitch J5 joint, and wrist roll J6 joint) are provided to improve the manipulability of the robotic arm. The axes of the wrist pitch J5 joint, the wrist roll J6 joint, and the forearm roll J4 joint are intersecting for reduced arm motion to reorient objects. The wrist roll J6 point is provided instead of two pitch joints in the wrist in order to improve object rotation.

### III. Example Combined UV Imaging and Irradiation Process

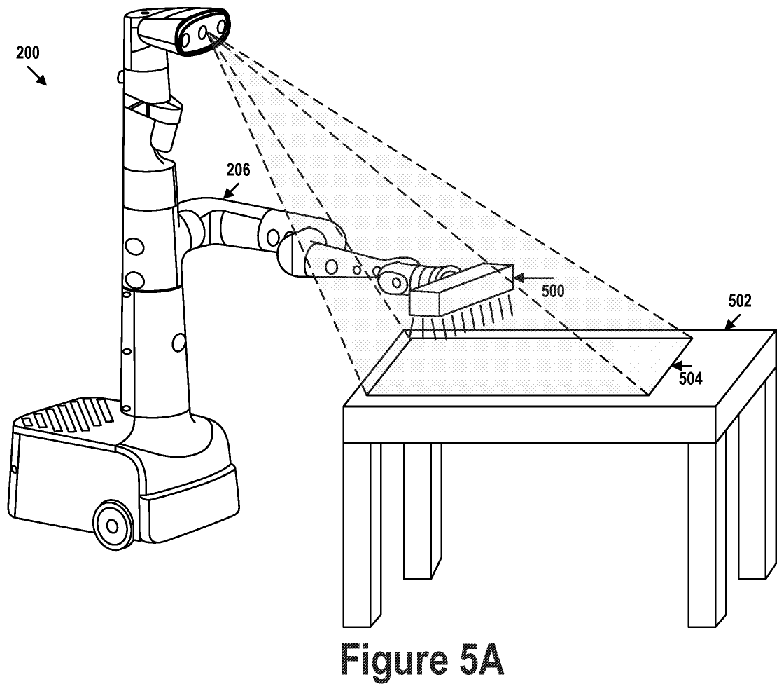

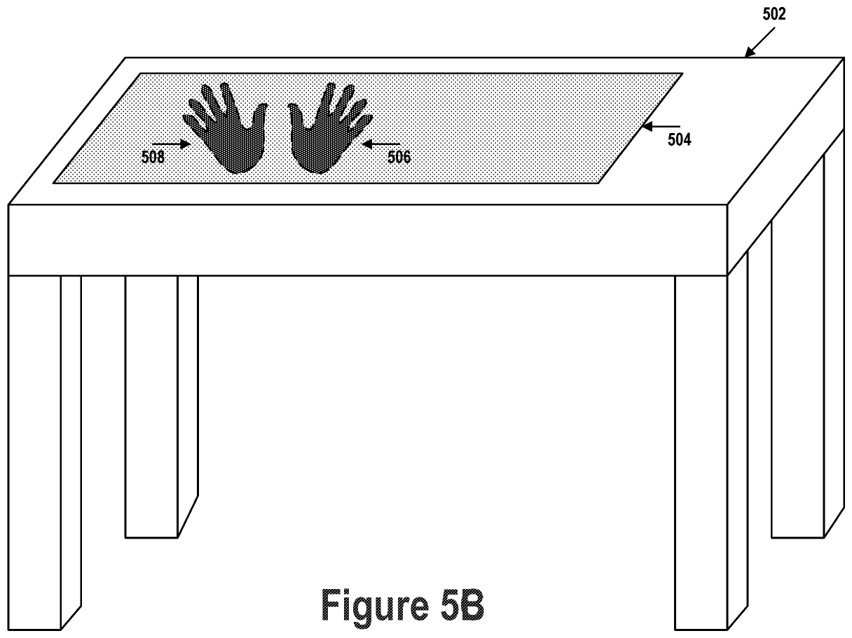

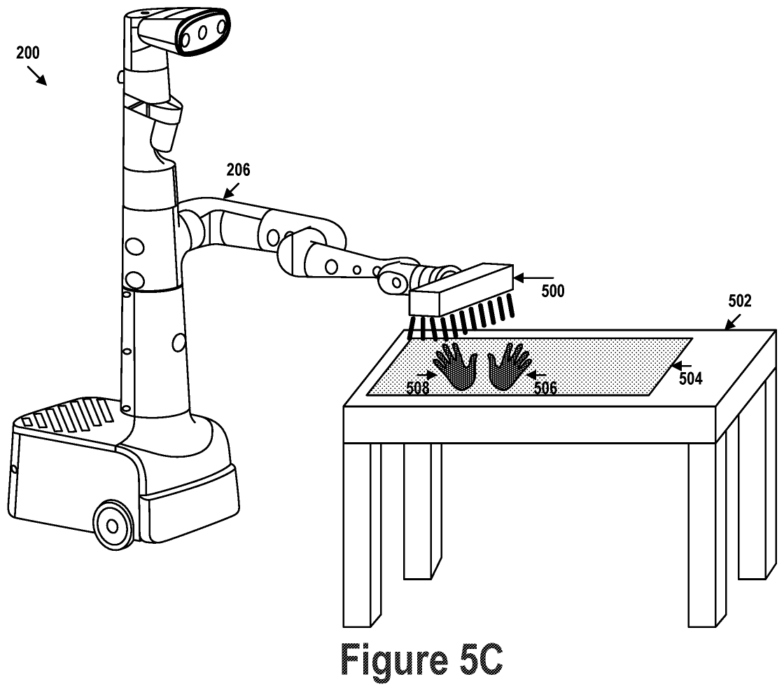

FIGS. 5A, 5B, and 5C illustrate an example scenario in which a UV illuminator is used to detect a stain (e.g., dirt, residue, foreign substance, or another undesirable material) on a feature of an environment, as well as to sanitize the detected stain. Specifically, FIG. 5A shows robot 200 operating in an environment that contains therein table 502. Robot 200 includes UV illuminator 500 connected to an end of arm 206. Thus, in the arrangement shown, EOAS 208 may include UV illuminator 500. Robot 200 is shown scanning a portion of table 502, as indicated by field of view 504, while UV illuminator 500 is used to emit light towards at least part of table 502, as indicated by the lines projected from the bottom of UV illuminator 500.

Field of view 504 may correspond to an image sensor configured to sense UV light and provided as part of perception suite 214. The UV image sensor may include components that are adapted for sensing UV light (e.g., wavelengths between 200 and 400 nanometers). For example, the UV image sensor may include one or more lenses, which may be made out of fused silica, fused quartz, and/or calcium fluoride, and thus configured to transmit UV light. The UV image sensor may also include optical filters configured to transmit the UV light and block non-UV light (e.g., visible light or infrared light). In some implementations, the UV image sensor may be disposed at a different location on robot 200, such as at the end of arm 206. Thus, in some cases, the UV image sensor and UV illuminator 500 may be co-located, may be simultaneously repositionable by way of arm 206, and/or may have the same or similar fields of view.

When imaged using the visible portion of the electromagnetic spectrum (e.g., wavelengths between 400 and 740 nanometers), the tabletop of table 502 may appear clean, as shown in FIG. 5A. Imaging the tabletop of table 502 using UV light, however, may reveal the presence of various stains on the tabletop of table 502. Specifically, some substances/materials may absorb UV light to a different extent than they absorb visible light, resulting in such substances/materials having a different appearance under UV illumination than under visible light illumination. For example, some substances/materials that would not appear in visible light images may appear in UV images.

Thus, FIG. 5B illustrates hand print 506 and hand print 508 defined on the tabletop of table 502. Hand prints 506 and 508 may be the result of a human actor touching table 502 and leaving behind some of the substances present on the actor's hands, such as bacteria, viruses, and/or sweat, among other possibilities. Hand prints 506 and 508 might not be apparent under visible light illumination, but may be visible in UV image data captured by the UV image sensor while UV illuminator 500 illuminates table 502. Thus, FIG. 5B may be understood to illustrate the content of such UV image data projected onto table 502, resulting in hand prints 506 and 508 being shown on table 502, although they might not be visible to the naked eye.

In addition to being used to detect hand prints 506 and 508, UV illuminator 500 may also be used to sanitize the portion of table 502 covered by hand prints 506 and 508. Specifically, a control system of robot 200 may be configured to process the UV image data to identify therein one or more visual patterns that represent actual and/or potential stains. The positions of these one or more visual patterns may be transformed from a reference frame of the UV image sensor to a reference frame of a map that represents the environment and is used by robot 200 to navigate through the environment. Thus, the respective positions of hand prints 506 and 508 may be represented within the map, and may thus be used to position UV illuminator 500 to perform the sanitization.

Sanitation of hand prints 506 and 508 may involve adjusting one or more parameters of UV illuminator 500 from a first set of values associated with imaging of the environment to a second set of values associated with sanitization of the environment. The one or more parameters may include a position of UV illuminator 500 relative to hand prints 506 and/or 508, a power with which UV illuminator 500 emits the UV light, a wavelength of the UV light emitted by UV illuminator 500, and/or a speed with which UV illuminator is moved relative to hand prints 506 and 508.

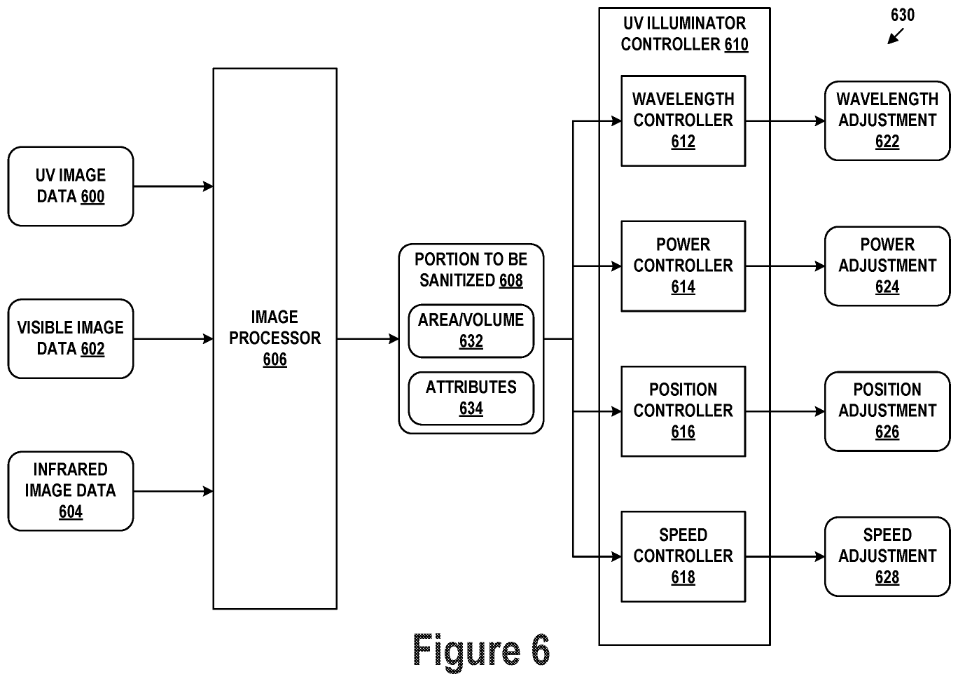

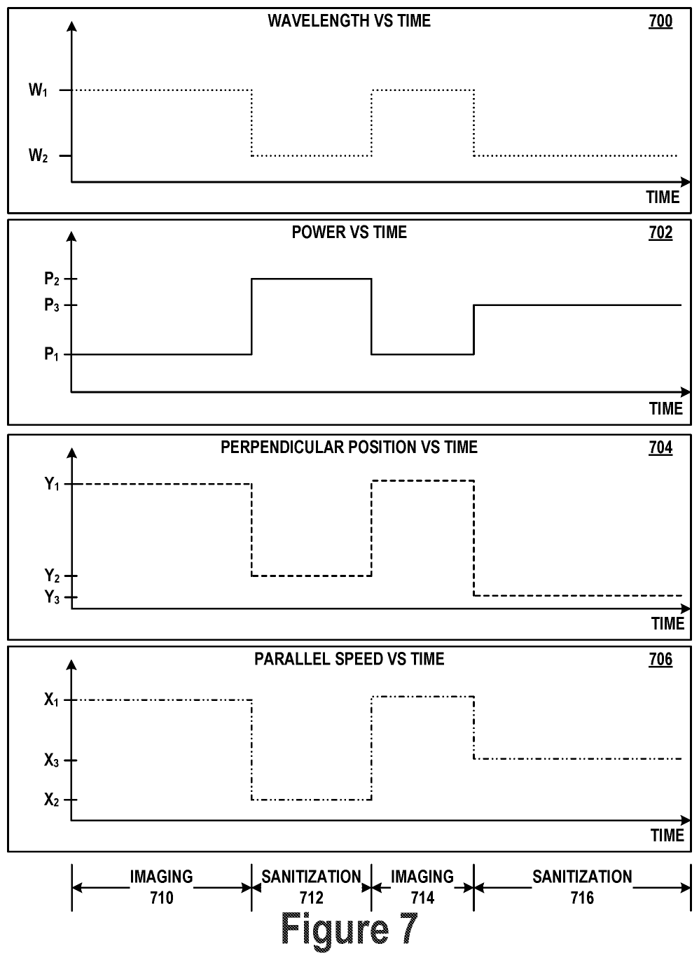

In general, aspects of UV imaging may be improved and/or optimized by using the first set of values for the one or more parameters, while aspects of sanitization may be improved and/or optimized by using the second set of values for the one or more parameters. Specifically, effective sanitization of a portion of the feature of the environment may involve the delivery of at least a threshold amount of energy per unit area (i.e., radiant exposure) at wavelengths that are effective at killing microorganisms. On the other hand, effective imaging of the portion of the feature may be associated with a smaller amount of energy per unit area and/or different wavelengths of UV light. Thus, each of the first set of values and the second set of values may be selected to deliver at least the corresponding amount of energy per unit area and/or the corresponding wavelength of UV light. Example adjustments are illustrated in and discussed in more detail with respect to FIGS. 6 and 7.

Once the one or more parameters are adjusted to reconfigure UV illuminator 500 for sanitization, robot 200 may move UV illuminator 500 relative to hand prints 506 and/or 508 (while UV illuminator 500 emits the UV light) to sanitize the corresponding portion of table 502, as illustrated in FIG. 5C. The darkened lines emitted out of UV illuminator 500 in FIG. 5C indicate that UV illuminator 500 is operated in accordance with the adjusted parameters. Specifically, by operating UV illuminator 500 in accordance with the adjusted one or more parameters, UV illuminator 500 may deliver to the area of space occupied by hand prints 506 and 508 a radiant exposure (i.e., energy per unit area) sufficient to kill microorganisms, thus sanitizing the corresponding portion of table 502.

In some implementations, prior to such sanitization by UV illuminator 500, robot 200 may be configured to manually clean hand prints 506 and/or 508. For example, robot 200 may include another arm equipped with a sponge, cloth, broom, sweeper, vacuum, sprayer, wiper, and/or other cleaning implement that may be used to clean hand prints 506 and/or 508 by physical manipulation of and/or interaction with table 502.

Additionally, based on and/or in response to sanitizing hand prints 506 and/or 508 using the UV light, the control system may update the map of the environment to indicate a time at which hand prints 506 and 508 have been cleaned. Specifically, sanitization of hand prints 506 and 508 with UV light might not produce a visually-perceptible result, and so hand prints 506 and 508, although now sanitized, may still appear the same way in additional UV image data. Accordingly, the map as updated may store the cleaning time of hand prints 506 and 508 and a representation of hand prints 506 and 508.

Thus, when robot 200 returns to table 502 at a later time, the control system may use the map to determine whether table 502 needs to be resanitized. Specifically, if additional UV image data captured at the later time indicates that the table contains thereof hand prints 506 and 508, but does not include additional stains, table 502 might not be resanitized. On the other hand, the additional UV image data captured at the later time indicates that the table contains thereon hand prints 506 and 508 and additional stains, then at least the additional stains on table 502 may be sanitized. In cases where hand prints 506 and/or 508 are also manually cleaned, the map might not be updated to indicate the cleaning/sanitization thereof, since manual cleaning may remove some or all traces of hand prints 506 and/or 508 such that these are not perceptible within the additional UV image data.

Such use of UV illuminator 500 for stain detection and sanitization may be particularly beneficial when paired with robot 200. Specifically, since human eyes are not adapted to see UV light, a human might not be able to use UV illuminator 500 to detect stains without also using a UV image sensor that is configured to sense the UV light. Additionally, even a human equipped with both UV illuminator 500 and the UV image sensor is unlikely to be able to accurately identify different types of stains, quickly adjust parameters of UV illuminator 500, and/or apply a consistent UV dose across different stains. Further, unlike a human whose skin and/or other organs may be affected by the UV light emitted by UV illuminator 500, robot 200 is very unlikely to be affected by the UV light, and may thus operate freely in the presence of such UV light.

Nevertheless, in some implementations, the systems and operations discussed herein may be used independently of robot 200. For example, UV illuminator 500 and/or the UV image sensor may be mounted at fixed locations within an environment (e.g., inside a vehicle), and the orientations thereof may be adjustable to project UV light at and sense UV light reflected from different portions of the environment. In other examples, UV illuminator 500 and/or the UV image sensor may be mounted to a predefined rail system or a manually-repositionable base (e.g., manually-repositionable by a human actor), and the orientations thereof may be adjustable.

In some implementations, the control system of robot 200 may be configured to use one or more other sensors provided on robot 200 (e.g., red-green-blue cameras, depth sensors, etc.) to identify actors, such as humans, dogs, and/or other robots, present within the environment. Based on and/or in response to identifying certain types of actors (e.g., humans or dogs), the control system may be configured to turn off UV illuminator 500 in order to avoid exposing these actors to the UV light. Thus, robot 200 may be configured to operate in the environment while certain types of actors are not around, and may pause the UV imaging and sanitization operations while these actors are present in the environment.

### IV. Example System for Controlling a UV Illuminator

FIG. 6 illustrates an example system that may be used to perform at least some of the operations described herein. Specifically, system 630 may include image processor 606 and UV illuminator controller 610, each of which may represent hardware (e.g., purpose-built circuitry), software (e.g., instructions configured to be executed by general-purpose circuitry), or a combination thereof. In some implementations, system 630 may form part of control system 118 of robotic system 100. Image processor 606 and UV illuminator controller 610 may be implemented using one or more rule-based algorithms and/or one or more machine learning algorithms/models.

Image processor 606 may be configured to process UV image data 600, which may correspond to the UV image data represented by field of view 504 in FIGS. 5A, 5B, and 5C, to identify portion to be sanitized 608 (i.e., portion 608). Thus, image processor 606 may include one or more image processing algorithms and/or machine learning algorithms/models configured to identify, within UV image data 600, one or more visual patterns associated with stains. The one or more visual patterns may include combinations of shapes, colors, and/or other visual features indicative of and/or associated with stains. For example, hand prints 506 and 508 of FIGS. 5B and 5C provide one example of a visual pattern that image processor 606 may be configured to detect.

Portion 608 may include a definition of area and/or volume 632 to be sanitized. Area/volume 632 may span at least a spatial extent of the region of the environment corresponding to any visual patterns detected by image processor 606. For example, area/volume 632 may indicate at least the respective area of the tabletop of table 502 spanned by hand prints 506 and 508. In some implementations, portion 608 may additionally indicate values of one or more attributes 634 of the stains to be sanitized. For example, attributes 634 may indicate a type of substance present in the portion to be sanitized, a source of the substance, and/or an amount of the substance, among other possibilities. Image processor 606 may be configured to determine and/or estimate values of attributes 634 based on the appearance of the corresponding portion of the environment within UV image data.

In some implementations, image processor 606 may additionally be configured to determine portion 608 based on visible image data 602 and/or infrared image data 604. Visible image data 602 and/or infrared image data 604 may represent additional stains and/or attributes thereof that might not be represented in UV image data 600, and might thus provide a more complete representation of the environment. In some cases, robot 200 may additionally operate based on depth data from one or more depth sensors, and such depth data may also be used by image processor 606 to facilitate identification of portion 608.

UV illumination controller 610 may include wavelength controller 612, power controller 614, position controller 616, and/or speed controller 618, each of which may be interconnected and configured to coordinate with one another. Based on portion 608, wavelength controller 612 may be configured to generate wavelength adjustment 622, power controller 614 may be configured to generate power adjustment 624, position controller 616 may be configured to generate position adjustment 626, and speed controller 618 may be configured to generate speed adjustment 628.

Each of wavelength adjustment 622, power adjustment 624, position adjustment 626, and speed adjustment 628 may represent an updated value for a corresponding parameter associated with UV illuminator 500. Specifically, wavelength adjustment 622 may indicate a wavelength range for the UV light emitted by UV illuminator 500. Power adjustment 624 may indicate a power with which UV illuminator 500 is to be driven. Position adjustment 626 may indicate a distance between UV illuminator 500 and the portion to be sanitized, and may be measured along a direction perpendicular to the portion to be sanitized. Speed adjustment 628 may indicate a speed with which UV illuminator is to be repositioned relative to the portion, and may be measured along a direction parallel to the portion to be sanitized.

Adjustments 622, 624, 626, and/or 628 may configure UV illuminator 500 to be used for sanitization, rather than imaging, of portion 608 of a feature of the environment. Once the sanitization is completed, controllers 612, 614, 616, and/or 616 may be configured to generate another set of adjustments (not shown) to configure UV illuminator 500 to be used for imaging, rather than sanitization, of additional portions of the environment.

Specifically, the parameters of UV illuminator 500 may include a wavelength of the UV light emitted by UV illuminator 500 towards portion 608 (e.g., hand prints 506 and/or 508). Imaging of portion 608 may involve emitting UV light having a first wavelength range (e.g., 300 to 400 nanometers), which the UV image sensor may detect relatively more efficiently than other wavelengths. Sanitization of portion 608 may involve emitting UV light having a second wavelength range (e.g., 200 to 300 nanometers), which may be more effective at killing microorganisms, and which may be represented by wavelength adjustment 622. Specifically, the germicidal properties of the UV light may peak between 255 and 275 nanometers (e.g., at 265 nanometers), due to these wavelengths being absorbed by and used to effectively damage the deoxyribonucleic acids (DNA) of the microorganisms. In one example, such a wavelength adjustment may be accomplished by adjusting one or more optical filters and/or modulators to block and/or transmit different wavelengths of light emitted by a broadband UV light source. In another example, such a wavelength adjustment may be accomplished by deactivating a first set of UV light emitting devices (LEDs) configured to emit UV light having the first wavelength range and activating a second set of UV LEDs configured to emit UV light having the second wavelength.

The parameters of UV illuminator 500 may additionally include a power with which UV illuminator 500 emits the UV light towards portion 608. Imaging of portion 608 may involve operating UV illuminator 500 at a first power level, resulting in a first number of photons emitted by UV illuminator 500 per unit time. Sanitization of portion 608 may involve operating UV illuminator 500 at a second higher power level, represented by power adjustment 624, resulting in a second higher number of photons emitted by UV illuminator 500 per unit time. Such a power adjustment may be accomplished by adjusting a voltage applied to UV illuminator 500 and/or by adjusting a duty cycle of a pulse-width-modulation (PWM) signal with which UV illuminator 500 is driven, among other possibilities.

The parameters of UV illuminator 500 may also include a distance between UV illuminator 500 and portion 608. Imaging of portion 608 may involve positioning UV illuminator 500 at a first distance relative to portion 608, while sanitization thereof may involve positioning the UV illuminator 500 at a second smaller distance, represented by bringing UV illuminator 500 closer to portion 608. Position adjustment 626 may represent the second smaller distance. In some cases, UV illuminator 500 may, as part of the sanitization process, be moved along a direction parallel to portion 608 in order to substantially uniformly “sweep” the UV light across portion 608. Thus, position adjustment 626 may be measured/defined along a direction perpendicular to portion 608 (e.g., perpendicular to the tabletop of table 502), and the position of UV illuminator 500 relative to portion 608 may remain constant as UV illuminator is ‘swept” across portion 608. Thus, depending on the shape of a surface of portion 608, the trajectory followed by UV illuminator 500 may be nonlinear in order to maintain the second distance relative to portion 608.

The parameter of UV illuminator 500 may further include a rate at which UV illuminator 500 is repositioned relative to portion 608. Imaging of portion 608 may allow UV illuminator 500 to be repositioned at a first speed relative to portion 608. The first speed may be defined at least in part by a frame rate at which the UV imager is able to capture the UV image data. Sanitization of portion 608 may involve repositioning UV illuminator 500 at a second speed relative to portion 608, and may be represented by speed adjustment 628. The second speed may be lower or higher than the first speed. In some cases, the speed with which UV illuminator 500 is repositioned may be defined along a direction parallel to portion 608, and may thus indicate the speed of the “sweep” of UV illuminator 500 relative to portion 608.

In practice, two or more of wavelength adjustment 622, power adjustment 624, position adjustment 626, and speed adjustment 628 of UV illuminator 500 may be made in coordination with one another. Specifically, as stated above, these parameters may be set so as to achieve a target amount of energy per unit area (i.e., target radiant exposure) to effectuate sanitization. In some cases, the target radiant exposure may vary as a function of the wavelength of the UV light emitted by UV illuminator 500. For example, the target radiant exposure for UV light having wavelength between 255 and 275 nanometers may be 0.5 millijoules per centimeter squared to 10 millijoule per centimeter squared (i.e., milliwatts/(centimeters²/seconds)), but the target radiant exposure for UV light with a different wavelength range may be different. In other cases, the target radiant exposure may vary as a function of attributes 634 of portion 608. For example, when the substance appears to sparsely/thinly cover portion 608, the target radiant exposure may be lower than when the substance appears to densely/thickly cover portion 608.

The radiant exposure delivered by UV illuminator 500 may be increased by bringing UV illuminator closer to hand prints 506 and 508, by increasing the power with which UV illuminator 500 emits the UV light, and/or by decreasing the speed with which UV illuminator 500 moves relative to portion 608. Thus, attributes 634 and wavelength adjustment 622 may indicate the target radiant exposure associated with effective sanitization, while power adjustment 624, position adjustment 626, and/or speed adjustment 628 may be selected to provide at least the target amount of energy per unit area.

Since robot 200 may be configured to accurately control the position and movement of UV illuminator 500 relative to features of the environment, robot 200 may be able to consistently apply at least the target radiant exposure as part of the sanitization process. Further, since the position and speed of UV illuminator 500 may be controlled by movement of arm 206, UV illuminator 500 may be brought relatively close to various features and/or swept relatively slowly with respect to the various features, thus allowing UV illuminator 500 to operate at a relatively low power while still providing at least the target radiant exposure as part of the sanitization process.

In some implementations, rather than using a dedicated UV image sensor to generate UV image data 600, UV image data 600 may instead be generated by way of a visible light image sensor (e.g., red-green-blue (RGB) camera). In one example, the power with which UV illuminator 500 emits the UV light may be increased to a point where the amount of UV light captured by the visible light image sensor sufficiently exceeds the amount of visible light captured thereby, resulting in the corresponding image data representing more UV light than visible light (e.g., (UV light)/(visible light)>1.0). In such cases, the power at which UV illuminator 500 is operated as part of sanitization may be lower than the power used for imaging.

In another example, the visible light image sensor may (while remaining stationary) be used to capture two images: one image with UV illumination and one image without UV illumination. The image without UV illumination may be subtracted from the image with UV illumination, resulting in a difference image that represents primarily the reflected UV light. In a further example, each color of a Bayer filter of the visible light image sensor may block UV light to a different extent. Thus, the signal generated by each pixel of the visible light image sensor may be weighted according to the color of its corresponding Bayer filter region. Specifically, when demosaicing the UV image from pixel data, pixels associated with color filters (e.g., filters configured to transmit green light) that are configured to transmit relatively more UV light may be weighted more heavily than pixels associated with color filters (e.g., filters configured to transmit red or blue light) that are configured to transmit relatively less UV light.

### V. Example UV Illuminator Parameter Adjustments

FIG. 7 illustrates a series of example adjustments made to parameters of UV illuminator 500 to transition UV illuminator 500 between imaging and sanitization modes. Specifically, FIG. 7 includes graph 700 that represents the wavelength of light emitted by UV illuminator 500 as a function of time, graph 702 that represents the power with which UV illuminator 500 is driven to emit the UV light as a function of time, graph 704 that represents the perpendicular position of UV illuminator 500 relative to the portion being sanitized as a function of time, and graph 706 that represents the parallel speed of UV illuminator 500 relative to the portion being sanitized as a function of time.

Each of graphs 700, 702, 704, and 706 is temporally-aligned along the horizontal axis, which is divided into imaging period 710, sanitization period 712, imaging period 714, and sanitization period 716. Periods 710, 712, 714, and 716 may have different durations. The duration of imaging periods 710 and 716 may be based on the number of UV image data frames captured before a portion to be sanitized is detected, among other factors. The duration of sanitization periods 712 and 714 may be based on a size of the portion to be sanitized, among other attributes thereof

Graph 700 illustrates that the wavelength of light emitted by UV illuminator 500 is set to a first wavelength value (or range) W₁ (e.g., 300 to 400 nanometers) during imaging periods 710 and 714, and is set to a second wavelength value (or range) W₂ (e.g., 200 to 300 nanometers) during sanitization periods 712 and 716.

Graph 702 illustrates that the power of UV illuminator 500 is set to a first power value P₁ during imaging periods 710 and 714, is set to a second power value P₂ during sanitization period 712, and is set to a third power value P₃ during sanitization period 716. The first power value P₁ is smaller than the third power value P₃ and the second power value P₂, and the third power value P₃ is smaller than the second power value P₂.

Graph 704 illustrates that the perpendicular position of UV illuminator 500 is set to a first position value Y₁ during imaging periods 710 and 714, is set to a second position value Y₂ during sanitization period 712, and is set to a third position value Y₃ during sanitization period 716. The first position value Y₁ is higher than the second position value Y₂ and the third position value Y₃, and the second position value Y₂ is higher than the third position value Y₃.

Graph 706 illustrates that the parallel speed of UV illuminator 500 is set to a first speed value V₁ during imaging periods 710 and 714, is set to a second speed value V₂ during sanitization period 712, and is set to a third speed value V₃ during sanitization period 716. The first speed value V₁ is higher than the second speed value V₂ and the third speed value V₃, and the third speed value V₃ is higher than the second speed value V₂.

The second power value P₂, the second position value Y₂, and the second speed value V₂ may be jointly determined to achieve at least the target radiant exposure associated with effective and/or optimal sanitization. Similarly, the third power value P₃, the third position value V₃, and the third speed value Y₃ may be jointly determined to achieve at least the target radiant exposure. Thus, although these two sets of values may differ, they may nevertheless result in application of at least the target radiant exposure to the portion of the feature to be sanitized. Further, by being able to jointly manipulate several different variables, the control system may be able to deliver the target radiant exposure while accounting for the operational context of robot 200 and/or attributes of the objects being sanitized. For example, fragile objects may be sanitized at higher power and slower speed from a larger distance, while sturdy objects may be sanitized at low power and high speed from a close distance.

### VI. Additional Example Operations

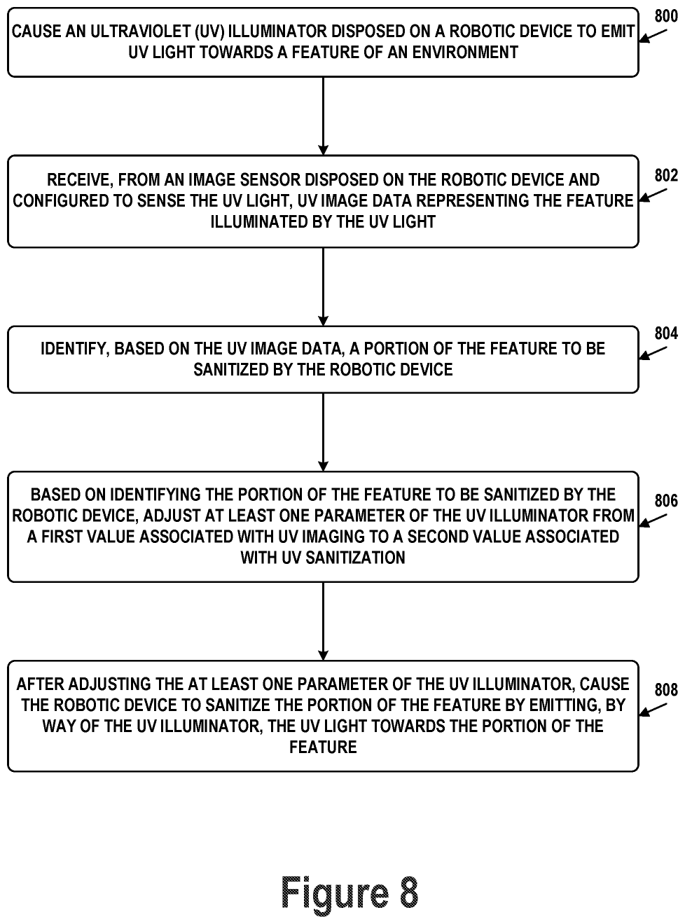

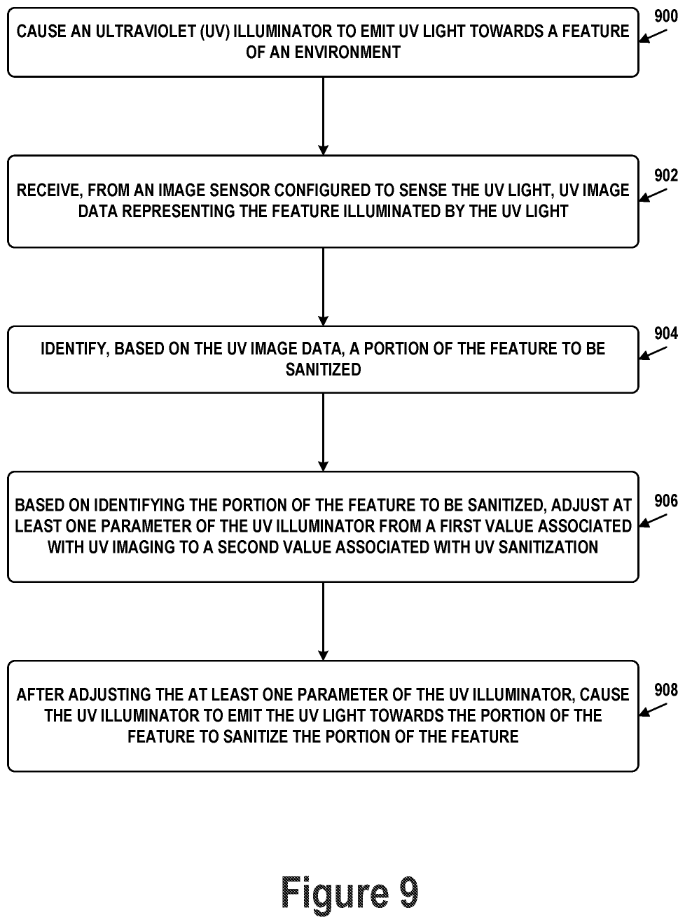

FIGS. 8 and 9 illustrate flow charts of operations related to detection and sanitization of stains using a UV illuminator. The operations may be carried out by robotic system 100, robot 200, system 630, and/or various other computing devices, among other possibilities. The embodiments of FIGS. 8 and/or 9 may be simplified by the removal of any one or more of the features shown therein. Further, these embodiments may be combined with features, aspects, and/or implementations of any of the previous figures or otherwise described herein.

Turning to FIG. 8, block 800 may involve causing a UV illuminator disposed on a robotic device to emit UV light towards a feature of an environment.

Block 802 may involve receiving, from an image sensor disposed on the robotic device and configured to sense the UV light, UV image data representing the feature illuminated by the UV light.

Block 804 may involve identifying, based on the UV image data, a portion of the feature to be sanitized by the robotic device.

Block 806 may involve, based on identifying the portion of the feature to be sanitized by the robotic device, adjusting at least one parameter of the UV illuminator from a first value associated with UV imaging to a second value associated with UV sanitization.

Block 808 may involve, after adjusting the at least one parameter of the UV illuminator, causing the robotic device to sanitize the portion of the feature by emitting, by way of the UV illuminator, the UV light towards the portion of the feature.

In some embodiments, the at least one parameter of the UV illuminator may be adjusted from the second value associated with UV sanitization to the first value associated with UV imaging based on the robotic device completing sanitization of the portion of the feature. After adjusting the at least one parameter of the UV illuminator from the second value associated with UV sanitization to the first value associated with UV imaging, additional UV image data representing additional features of the environment illuminated by the UV light may be received from the image sensor.

In some embodiments, identifying the portion of the feature to be sanitized by the robotic device may include determining that the portion of the feature has been touched by an actor by detecting one or more visual patterns within the UV image data.

In some embodiments, a type of substance present on the portion of the feature may be determined based on a visual appearance of the portion of the feature within the UV image data. The second value associated with UV sanitization may be selected based on the type of substance present on the portion of the feature.

In some embodiments, a map of the environment may be configured to track portions of features of the environment that have been previously sanitized. Identifying the portion of the feature to be sanitized by the robotic device may include determining, based on the map of the environment, that the portion of the environment has not been sanitized within a preceding predetermined period of time. After the robotic device sanitizes the portion of the feature by emitting the UV light towards the portion of the feature, the map of the environment may be updated to indicate a time at which the portion of the feature has been sanitized by the robotic device.

In some embodiments, the at least one parameter of the UV illuminator may include a power level with which the UV illuminator emits the UV light. The first value may include a first power level, the second value may include a second power level, and the second power level may be higher than the first power level.

In some embodiments, the at least one parameter of the UV illuminator may include a position of the UV illuminator relative to the portion of the feature. The first value may include a first position relative to the portion of the feature, the second value may include a second position relative to the portion of the feature, and the second position may be closer to the portion of the feature than the first position.

In some embodiments, the at least one parameter of the UV illuminator may include a movement speed of the UV illuminator relative to the portion of the feature. The first value may include a first movement speed relative to the portion of the feature, the second value may include a second movement speed relative to the portion of the feature, and the second movement speed may be smaller than the first movement speed.

In some embodiments, the at least one parameter of the UV illuminator may include a wavelength of the UV light emitted by the UV illuminator. The first value may include a first wavelength range, and the second value may include a second wavelength range.

In some embodiments, the second wavelength range may include wavelengths between 255 nanometers and 275 nanometers.

In some embodiments, the UV light may include wavelength between 200 nanometers and 300 nanometers, and the image sensor may be configured to detect wavelengths between 200 nanometers and 300 nanometers.

In some embodiments, the UV light may include wavelength between 200 nanometers and 400 nanometers, and the image sensor may be configured to detect wavelengths between 300 nanometers and 400 nanometers.

In some embodiments, the UV illuminator may be connected to an arm of the robotic device. Thus, the UV illuminator may be repositionable relative to the environment by way of the arm.

In some embodiments, an additional image sensor disposed on the robotic device may be configured to sense additional light other than the UV light. Additional image data representing the feature of the environment illuminated by the additional light may be received from the additional image sensor. An additional portion of the feature to be sanitized by the robotic device may be identified based on the additional image data. The at least one parameter of the UV illuminator may be adjusted from the first value associated with UV imaging to the second value associated with UV sanitization further based on identifying the additional portion of the feature to be sanitized by the robotic device. After adjusting the at least one parameter of the UV illuminator, the robotic device may be caused to sanitize the additional portion of the feature by emitting, by way of the UV illuminator, the UV light towards the additional portion of the feature.

In some embodiments, an additional image sensor disposed on the robotic device may be configured to sense additional light other than the UV light. Additional image data representing the feature of the environment illuminated by the additional light may be received from the additional image sensor. The portion of the feature to be sanitized by the robotic device may be identified further based on the additional image data.

In some embodiments, prior to causing the robotic device to sanitize the portion of the feature by emitting the UV light towards the portion of the feature, the robotic device may be caused to manually clean the portion of the feature by interacting with the portion of the feature by way of an arm of the robotic device.

In some embodiments, a type of substance present on the portion of the feature may be determined based on a visual appearance of the portion of the feature within the UV image data. Based on the type of substance present on the portion of the feature, a first end effector may be selected from a plurality of end effectors provided on the arm of the robotic device. The robotic device may be caused to manually clean the portion of the feature using the first end effector.

Turning to FIG. 9, Block 900 may involve causing a UV illuminator to emit UV light towards a feature of an environment.

Block 902 may involve receiving, from an image sensor configured to sense the UV light, UV image data representing the feature illuminated by the UV light.

Block 904 may involve identifying, based on the UV image data, a portion of the feature to be sanitized.

Block 906 may involve, based on identifying the portion of the feature to be sanitized, adjusting at least one parameter of the UV illuminator from a first value associated with UV imaging to a second value associated with UV sanitization.

Block 908 may involve, after adjusting the at least one parameter of the UV illuminator, causing the UV illuminator to emit the UV light towards the portion of the feature to sanitize the portion of the feature.

In some embodiments, the at least one parameter of the UV illuminator may be adjusted from the second value associated with UV sanitization to the first value associated with UV imaging based on completing sanitization of the portion of the feature. After adjusting the at least one parameter of the UV illuminator from the second value associated with UV sanitization to the first value associated with UV imaging, additional UV image data representing additional features of the environment illuminated by the UV light may be received from the image sensor.

In some embodiments, identifying the portion of the feature to be sanitized may include determining that the portion of the feature has been touched by an actor by detecting one or more visual patterns within the UV image data.

In some embodiments, a type of substance present on the portion of the feature may be determined based on a visual appearance of the portion of the feature within the UV image data. The second value associated with UV sanitization may be selected based on the type of substance present on the portion of the feature.

In some embodiments, a map of the environment may be configured to track portions of features of the environment that have been previously sanitized. Identifying the portion of the feature to be sanitized may include determining, based on the map of the environment, that the portion of the environment has not been sanitized within a preceding predetermined period of time. After the portion of the feature is sanitized by emitting the UV light towards the portion of the feature, the map of the environment may be updated to indicate a time at which the portion of the feature has been sanitized.

In some embodiments, the at least one parameter of the UV illuminator may include a power level with which the UV illuminator emits the UV light. The first value may include a first power level, the second value may include a second power level, and the second power level may be higher than the first power level.

In some embodiments, the at least one parameter of the UV illuminator may include a position of the UV illuminator relative to the portion of the feature. The first value may include a first position relative to the portion of the feature, the second value may include a second position relative to the portion of the feature, and the second position may be closer to the portion of the feature than the first position.

In some embodiments, the at least one parameter of the UV illuminator may include a movement speed of the UV illuminator relative to the portion of the feature. The first value may include a first movement speed relative to the portion of the feature, the second value may include a second movement speed relative to the portion of the feature, and the second movement speed may be smaller than the first movement speed.

In some embodiments, the at least one parameter of the UV illuminator may include a wavelength of the UV light emitted by the UV illuminator. The first value may include a first wavelength range, and the second value may include a second wavelength range.

In some embodiments, the second wavelength range may include wavelengths between 255 nanometers and 275 nanometers.

In some embodiments, the UV light may include wavelength between 200 nanometers and 300 nanometers, and the image sensor may be configured to detect wavelengths between 200 nanometers and 300 nanometers.

In some embodiments, the UV light may include wavelength between 200 nanometers and 400 nanometers, and the image sensor may be configured to detect wavelengths between 300 nanometers and 400 nanometers.

In some embodiments, the UV illuminator may be disposed at a fixed location within the environment. The orientation of the UV illuminator relative to the environment may be adjustable.

In some embodiments, an additional image sensor may be configured to sense additional light other than the UV light. Additional image data representing the feature of the environment illuminated by the additional light may be received from the additional image sensor. An additional portion of the feature to be sanitized may be identified based on the additional image data. The at least one parameter of the UV illuminator may be adjusted from the first value associated with UV imaging to the second value associated with UV sanitization further based on identifying the additional portion of the feature to be sanitized. After adjusting the at least one parameter of the UV illuminator, the UV illuminator may be caused to emit the UV light towards the additional portion of the feature to sanitize the additional portion of the feature.

In some embodiments, an additional image sensor may be configured to sense additional light other than the UV light. Additional image data representing the feature of the environment illuminated by the additional light may be received from the additional image sensor. The portion of the feature to be sanitized may be identified further based on the additional image data.

In some embodiments, prior to causing the UV illuminator to emit the UV light towards the portion of the feature to sanitize the portion of the feature, the portion of the feature may be manually cleaned.

### VII. Conclusion

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. In the figures, similar symbols typically identify similar components, unless context dictates otherwise. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information may correspond to circuitry that can be configured to perform the specific logical functions of a herein-described method or technique. Alternatively or additionally, a block that represents a processing of information may correspond to a module, a segment, or a portion of program code (including related data). The program code may include one or more instructions executable by a processor for implementing specific logical operations or actions in the method or technique. The program code and/or related data may be stored on any type of computer readable medium such as a storage device including random access memory (RAM), a disk drive, a solid state drive, or another storage medium.

The computer readable medium may also include non-transitory computer readable media such as computer readable media that store data for short periods of time like register memory, processor cache, and RAM. The computer readable media may also include non-transitory computer readable media that store program code and/or data for longer periods of time. Thus, the computer readable media may include secondary or persistent long term storage, like read only memory (ROM), optical or magnetic disks, solid state drives, compact-disc read only memory (CD-ROM), for example. The computer readable media may also be any other volatile or non-volatile storage systems. A computer readable medium may be considered a computer readable storage medium, for example, or a tangible storage device.

Moreover, a step or block that represents one or more information transmissions may correspond to information transmissions between software and/or hardware modules in the same physical device. However, other information transmissions may be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. A system comprising:
a robotic device;
an ultraviolet (UV) illuminator disposed on the robotic device and configured to emit UV light;
an image sensor disposed on the robotic device and configured to sense the UV light; and
circuitry configured to perform operations comprising:
causing the UV illuminator to emit the UV light towards a feature of an environment;
receiving, from the image sensor, UV image data representing the feature illuminated by the UV light;
identifying, based on the UV image data, a portion of the feature to be sanitized by the robotic device;
based on identifying the portion of the feature to be sanitized by the robotic device, adjusting at least one parameter of the UV illuminator from a first value associated with UV imaging to a second value associated with UV sanitization; and
after adjusting the at least one parameter of the UV illuminator, causing the robotic device to sanitize the portion of the feature by emitting, by way of the UV illuminator, the UV light towards the portion of the feature.

2. The system of claim 1, wherein the operations further comprise:
based on the robotic device completing sanitization of the portion of the feature, adjusting the at least one parameter of the UV illuminator from the second value associated with UV sanitization to the first value associated with UV imaging; and
after adjusting the at least one parameter of the UV illuminator from the second value associated with UV sanitization to the first value associated with UV imaging, receiving, from the image sensor, additional UV image data representing additional features of the environment illuminated by the UV light.

3. The system of claim 1, wherein identifying the portion of the feature to be sanitized by the robotic device comprises:
determining that the portion of the feature has been touched by an actor by detecting one or more visual patterns within the UV image data.

4. The system of claim 1, wherein the operations further comprise:
determining, based on a visual appearance of the portion of the feature within the UV image data, a type of substance present on the portion of the feature; and
selecting the second value associated with UV sanitization based on the type of substance present on the portion of the feature.

5. The system of claim 1, wherein a map of the environment is configured to track portions of features of the environment that have been previously sanitized, and wherein identifying the portion of the feature to be sanitized by the robotic device comprises:
determining, based on the map of the environment, that the portion of the environment has not been sanitized within a preceding predetermined period of time, wherein the operations further comprise:
after the robotic device sanitizes the portion of the feature by emitting the UV light towards the portion of the feature, updating the map of the environment to indicate a time at which the portion of the feature has been sanitized by the robotic device.

6. The system of claim 1, wherein the at least one parameter of the UV illuminator comprises a power level with which the UV illuminator emits the UV light, wherein the first value comprises a first power level, wherein the second value comprises a second power level, and wherein the second power level is higher than the first power level.

7. The system of claim 1, wherein the at least one parameter of the UV illuminator comprises a position of the UV illuminator relative to the portion of the feature, wherein the first value comprises a first position relative to the portion of the feature, wherein the second value comprises a second position relative to the portion of the feature, and wherein the second position is closer to the portion of the feature than the first position.

8. The system of claim 1, wherein the at least one parameter of the UV illuminator comprises a movement speed of the UV illuminator relative to the portion of the feature, wherein the first value comprises a first movement speed relative to the portion of the feature, wherein the second value comprises a second movement speed relative to the portion of the feature, and wherein the second movement speed is smaller than the first movement speed.

9. The system of claim 1, wherein the at least one parameter of the UV illuminator comprises a wavelength of the UV light emitted by the UV illuminator, wherein the first value comprises a first wavelength range, and wherein the second value comprises a second wavelength range.

10. The system of claim 9, wherein the second wavelength range includes wavelengths between 255 nanometers and 275 nanometers.

11. The system of claim 1, wherein the UV light comprises wavelength between 200 nanometers and 300 nanometers, and wherein the image sensor is configured to detect wavelengths between 200 nanometers and 300 nanometers.

12. The system of claim 1, wherein the UV light comprises wavelength between 200 nanometers and 400 nanometers, and wherein the image sensor is configured to detect wavelengths between 300 nanometers and 400 nanometers.

13. The system of claim 1, wherein the UV illuminator is connected to an arm of the robotic device, and wherein the UV illuminator is repositionable relative to the environment by way of the arm.

14. The system of claim 1, further comprising:
an additional image sensor disposed on the robotic device and configured to sense additional light other than the UV light, wherein the operations further comprise:
receiving, from the additional image sensor, additional image data representing the feature of the environment illuminated by the additional light;
identifying, based on the additional image data, an additional portion of the feature to be sanitized by the robotic device;
adjusting the at least one parameter of the UV illuminator from the first value associated with UV imaging to the second value associated with UV sanitization further based on identifying the additional portion of the feature to be sanitized by the robotic device; and
after adjusting the at least one parameter of the UV illuminator, causing the robotic device to sanitize the additional portion of the feature by emitting, by way of the UV illuminator, the UV light towards the additional portion of the feature.

15. The system of claim 1, further comprising:
an additional image sensor disposed on the robotic device and configured to sense additional light other than the UV light, wherein the operations further comprise:
receiving, from the additional image sensor, additional image data representing the feature of the environment illuminated by the additional light, wherein the portion of the feature to be sanitized by the robotic device is identified further based on the additional image data.

16. The system of claim 1, wherein the operations further comprise:
prior to causing the robotic device to sanitize the portion of the feature by emitting the UV light towards the portion of the feature, causing the robotic device to manually clean the portion of the feature by interacting with the portion of the feature by way of an arm of the robotic device.

17. The system of claim 16, wherein the operations further comprise:
determining, based on a visual appearance of the portion of the feature within the UV image data, a type of substance present on the portion of the feature; and
selecting, based on the type of substance present on the portion of the feature, a first end effector from a plurality of end effectors provided on the arm of the robotic device, wherein the robotic device is caused to manually clean the portion of the feature using the first end effector.

18. A computer-implemented method comprising:
causing an ultraviolet (UV) illuminator disposed on a robotic device to emit UV light towards a feature of an environment;
receiving, from an image sensor disposed on the robotic device and configured to sense the UV light, UV image data representing the feature illuminated by the UV light;
identifying, based on the UV image data, a portion of the feature to be sanitized by the robotic device;
based on identifying the portion of the feature to be sanitized by the robotic device, adjusting at least one parameter of the UV illuminator from a first value associated with UV imaging to a second value associated with UV sanitization; and
after adjusting the at least one parameter of the UV illuminator, causing the robotic device to sanitize the portion of the feature by emitting, by way of the UV illuminator, the UV light towards the portion of the feature.

19. The computer-implemented method of claim 18, wherein identifying the portion of the feature to be sanitized by the robotic device comprises:
determining that the portion of the feature has been touched by an actor by detecting one or more visual patterns within the UV image data.

20. A non-transitory computer-readable medium having stored thereon instructions that, when executed by a computing device, cause the computing device to perform operations comprising:
causing an ultraviolet (UV) illuminator disposed on a robotic device to emit UV light towards a feature of an environment;
receiving, from an image sensor disposed on the robotic device and configured to sense the UV light, UV image data representing the feature illuminated by the UV light;
identifying, based on the UV image data, a portion of the feature to be sanitized by the robotic device;
based on identifying the portion of the feature to be sanitized by the robotic device, adjusting at least one parameter of the UV illuminator from a first value associated with UV imaging to a second value associated with UV sanitization; and
after adjusting the at least one parameter of the UV illuminator, causing the robotic device to sanitize the portion of the feature by emitting, by way of the UV illuminator, the UV light towards the portion of the feature.
